(19) United States
(12) Patent Application Publication
(10) Pub. No.: US 2026/0063613 A1
(43) Pub. Date: Mar. 5, 2026

(54) METHOD FOR DETERMINING SOIL CARBON CONTENT

(71) Applicant: Nutrien AG Solutions, Inc., Loveland, CO (US)

(72) Inventors: Guy Tal, Cooper City, FL (US); Nathaniel Levitan, Loveland, CO (US); Keyvan Malek, Champaign, IL (US)

(21) Appl. No.: 18/823,142

(22) Filed: Sep. 3, 2024

(51) Int. Cl. G01N 33/24; G01N 1/08

(52) U.S. Cl. CPC G01N 33/245; G01N 1/08

(57) ABSTRACT

A method for calculating total organic carbon (TOC) for a sample location includes obtaining an intact core soil sample from a sample location, the core soil sample having a depth and mass; transferring a predetermined fixed mass M from the intact core soil sample into an analysis sample container to obtain an analysis sample; and providing a subsample of the analysis sample for soil organic carbon (SOC) concentration testing.

### FIELD

This disclosure relates generally to a method for measuring carbon content of soil. More specifically, this disclosure relates to a method for measuring soil carbon content for agricultural crop ground.

### BACKGROUND

Carbon sequestration refers to processes that include capturing and storing carbon dioxide (CO₂) and other forms of carbon. Incentives for carbon sequestration include its ability to mitigate climate change. Economic incentives, such as a carbon credit market, are being developed to incentivize growers to use agricultural practices that produce carbon sequestration. Accurate measurements of soil carbon quantities facilitate accurate quantification of changes in carbon quantities over time. It is believed that measuring soil organic carbon (SOC) stock, which is defined as the amount of organic carbon in the soil per unit land area, accurately and at a low cost is important to developing a robust carbon credit market.

Known approaches for measuring the amounts of carbon in soil include collecting samples of the soil of interest (e.g., in an agricultural field), and processing the soil samples. Previously, soil samples for carbon sampling were collected by using soil sample probes in the form of cylindrical tubes that are inserted into the soil to predetermined depths and withdrawn with a predetermined sample volume. The sample probe was then emptied into a sample container and sent to a laboratory for analysis. This type of sampling can be referred to as a fixed depth sampling method. In some processes, the soil sample was from a discrete or single location. In other methods, the soil sample was an aggregate sample formed by sample probes from more than one location. Because it is generally cost prohibitive to directly measure the mass of organic carbon of the entire sample, the total organic carbon of the sample is generally determined by measuring the SOC concentration of a small subsample and multiplying it by the mass of the sample. The subsample analyzed for SOC concentration needs to be representative of the entire sample which is approximated by a homogenization step. The homogenization step is complicated both procedurally and scientifically by coarse fragments. Procedurally, the coarse fragments must be removed as they are not considered part of soil for carbon measurements. Scientifically, differences in coarse fragment depths between samples can affect the representativeness of the subsample.

Soil is a complex structure and is comprised of a plurality of layers. Each layer can have different properties including density and carbon content. Additionally, the carbon content of a portion of soil closer to the surface is generally higher than a portion of soil further from the surface (i.e., at a greater depth). For example, typically the carbon concentration at 5 cm depth is greater than the concentration at 25 cm depth. Thus, valuable information is lost if the layers are not maintained in a soil sample and/or the sample is not representative of each layer.

Additionally, determining SOC stock using a fixed depth method may not be sufficiently accurate because farm practices change the bulk density of the soil over time. For example, a tractor or human traffic can compact the soil. Since the density of the soil is now more compact, there is more soil in the core when using a fixed depth method. Additionally, changes in the organic matter, for example from crop residue, can also naturally change the bulk density of the soil. For these and other reasons, a new method to accurately measure or determine the amounts of carbon present in agricultural soils is needed.

### SUMMARY

In Example 1, a method for calculating total organic carbon (TOC) for a sample location includes obtaining an intact core soil sample from a sample location, the core soil sample having a depth and mass; transferring a predetermined fixed mass M from the intact core soil sample into an analysis sample container to obtain an analysis sample; and providing a subsample of the analysis sample for soil organic carbon (SOC) concentration testing.

In Example 2, the method of Example 1, wherein the predetermined mass is based on a soil characterization of the sample location.

In Example 3, the method of Example 2, wherein the soil characterization is soil bulk density, and wherein the predetermined fixed mass M from a sample location having a high soil bulk density is PM1 and the predetermined fixed mass M from a sample location having a low soil bulk density is PM2, where PM1 and PM2 are different.

In Example 4, the method of Example 2, wherein the soil characterization is geographic location, and wherein the predetermined fixed mass M from a first geographical location is PM1 and the predetermined fixed mass M from a second geographical location is PM2, where PM1 and PM2 are different.

In Example 5, the method of Example 4, wherein a soil database is used to convert the geographic location to a soil bulk density category.

In Example 6, the method of Example 1, wherein the depth of the core soil sample is from about 35 centimeters (cm) to about 45 cm.

In Example 7, the method of Example 1, wherein the depth of the core soil sample is based on a soil characteristic of the sample location.

In Example 8, the method of Example 7, wherein the soil characteristic is soil bulk density.

In Example 9, the method of Example 1, wherein the depth of the core soil sample is based on the geographic location of the sample location.

In Example 10, the method of Example 1 wherein if the mass of intact core soil sample is less than the predetermined fixed mass M, the predetermined fixed mass M is the mass of the intact core sample.

In Example 11, the method of Example 1, wherein if the predetermined fixed mass M is obtained with less than a depth DS, the depth DS from the intact core soil sample is transferred to the analysis sample container with sample mass SM, where DS is the soil depth sufficient to characterize changes in soil carbon due to agriculture practices,

In Example 12, the method of Example 11, wherein DS is 30 cm.

In Example 13, the method of Example 1 and further including the step of calculating the TOC by multiplying predetermined fixed mass M by a SOC concentration from the SOC concentration testing.

### BRIEF DESCRIPTION OF THE DRAWINGS

FIG. 1 is a block diagram illustrating a method for calculating soil organic carbon (SOC).

### DETAILED DESCRIPTION

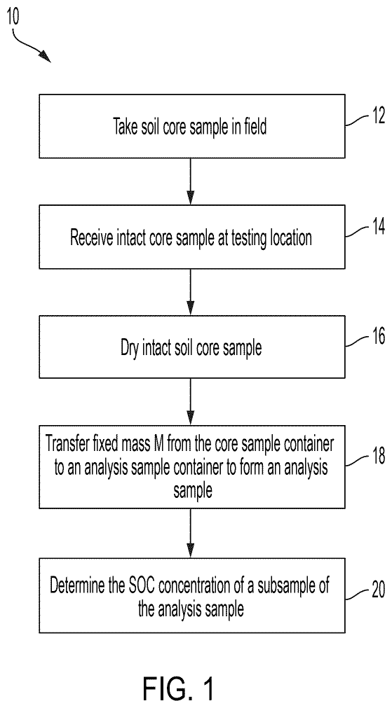

FIG. 1 is a block diagram illustrating method 10 for calculating soil organic carbon (SOC) using a mass equivalence approach. Method 10 includes taking a soil core sample in a field (step 12), receiving the intact core sample in a sample container at a testing location (step 14), drying the intact core sample (step 16), transferring fixed mass M from the core sample container to an analysis sample container to form an analysis sample (step 18) and determining the SOC concentration of a subsample of the analysis sample (step 20).

In step 12 a soil core sample is taken at the location of interest. In some examples, the location of interest is an agricultural field, and the SOC analysis can be used as part of a carbon sequestration program. For example, the location of interest may be a field for growing row or other types of crops for human and/or animal consumption. The soil core sample is taken such that sufficient soil is obtained for the SOC testing. Additionally, the soil core sample is taken in a manner that retains the soil layers. In one example, a core of a given length is extracted from the soil into a first container or sample container. For example, a hydraulic system can be used to press a soil probe into the ground to collect the soil core sample in a soil probe. In some embodiments, the soil probe may be slotted to allow for visual inspection of the soil core sample. In some prior sampling protocols, soil samples are taken to a depth of 30 centimeters (cm.) In some examples of the current invention, the soil core sample is taken to a greater depth, for example to 35 cm, 40 cm, 45 cm, 50 cm or 55 cm or any range thereof. The hydraulic press can be used to collect a deeper intact sample than may be possible using a hand sampling probe. The length and diameter of the core sample determine the volume and thus the mass of the sample. In some embodiments, the soil core sample may be between about 1 inch (2.54 cm) and 3 inches (7.62 cm) in diameter, such as between about 1.5 and 2.5 inches (3.81 and 6.35 cm) or about 2 inches (5.08 cm), including for example 1.9 inches (4.83 cm).

The soil core sample is sent intake to a testing location for further preparation and/or analysis. In some embodiments, the soil core sample is sent in the sample container, such as the sample probe, which is used to extract the soil core sample from the ground. In other embodiments, the soil core sample can be removed from the sample container used for the extraction and sent in a different sample container. The sample container is designed so that the soil core sample arrives at the testing location intact. For example, the soil core sample can be at least partially dried in the soil probe and then the intact core is slid into a close-fitting sleeve, such as a plastic sleeve, for transportation. In some examples, the soil core sample can be delivered to the testing location by an individual, such as the individual that gathered the sample. In other examples, the soil core sample can be shipped to the testing location using commercial shipping or courier. In each of these examples, it is important that the soil core sample is packaged such that the soil layers remain substantially intact in a single sample. In this way, the soil core sample in the sample container contains layers which are representative of those of the original sample site.

In some examples, a pre-filter can be used to determine the size (e.g., depth) of the soil core sample before the sample is collected. The pre-filter can be based on a soil characterization of the sample location. Example pre-filters include by geographic boundary, such as state of the United States, county or borough, or region, soil bulk density or a combination of both. For example, based on geographic data, the sample area can be characterized based on a soil characteristic and the sample depth determined based on this characteristic. In one example, the sample depth is determined based on bulk density and the sample depth for a field identified as having high density soil will be one specified depth while the sample size for a field identified as having low density soil will be a different specified depth.

In step 14, the intact soil core sample in the sample container is received at or obtained by the testing location. As used herein, this means that the soil core sample is in substantially the same form as when removed from the soil such that it retains layers which are representative (i.e., at the same location) of those of the original sample site. The testing location can be a soil analysis laboratory or a site which prepares samples for soil test analysis, such as carbon organic soil concentration testing. In some embodiments, the intact soil core sample may be weighed prior to step 18.

In step 16, the soil core sample in the sample container is dried to remove all or substantially all of the moisture in the sample. One skilled in the art will recognize how to properly dry the sample.

In step 18, at least a portion of the soil is transferred or poured from the intact soil core sample in the first container into a second container for an analysis sample. A predetermined fixed mass M of the soil from the soil core sample is poured into the second container. The soil is gently poured into the second container so that fixed matter M is reached with the soil closest to surface. A scale can be used to measure the mass transferred to the second container and pouring is stopped when the mass M is reached.

The soil can be poured such that the soil that was originally closer to the surface is poured before soil that was deeper in the ground. In this way, the analysis sample contains a specified mass that is representative of the soil depth. In contrast, if the soil core sample were mixed and the layers not retained, the mass would be representative of the sample overall and information specific to the layers would be lost. Carbon content is not consistent through the soil. For example, carbon content of certain soil layers, such as those closer to the surface, may be higher than soil that is deeper in the ground. When the soil layers are not maintained (e.g., the soil sample is mixed and a specified mass is used from the mixed sample) and the entire soil core sample is not needed to reach fixed mass M, the generally lower carbon content of the deeper layers dilute the carbon concentration of the sample, and the SOC measurement is not accurate.

The mass poured into the second container for the analysis sample is a predetermined mass. The predetermined fixed mass M may vary based on a soil characteristic and/or geographic location of the sample site. In some embodiments, the predetermined fixed mass M is selected to allow the greatest number of sites to have the standard mass M falling between 30 cm and 45 cm of the core sample. This minimizes the number of failed samples as discussed below. In one example, the predetermined fixed mass is based on the soil bulk density. For example, based on geographical location, the sample location can be categorized as high soil bulk density or low soil bulk density. In this embodiment, the predetermined fixed mass for the high soil bulk density location is PM1 and the predetermined fixed mass for the low soil bulk density location is PM2, where PM1 and PM2 are different. For example, PM1 can be less than PM2. In another example, the predetermined fixed mass M is based on geographical location. For example, all sample locations within a certain state or county can be predetermined fixed mass PM1 and all sample locations within a different state or county can be predetermined fixed mass PM2, where PM1 and PM2 are different. In some embodiments, the predetermined fixed mass M is between about 600 grams and about 1,000 grams or between about 700 grams and about 900 grams or between about 800 grams and about 900 grams.

In some embodiments, the geographic location is a precise location. In other embodiments, it can be the average for a geographical area, such as a state, a county or a geographic region such as the mid-western region of the United States. In some embodiments, a soil database can be used to make the link between geography and the expected soil type and/or soil bulk density. For example, the predetermined fixed mass M can be determined for a given geographic location by using a soil database to determine the expected soil bulk density at that location. The expected soil bulk density can then be used to select the predetermined fixed mass M. In some embodiments, the expected soil bulk density value can be categorized as low soil bulk density or high soil bulk density as discussed above based on precise geography location, or geographic region, such as state, county or region (i.e., mid-west of the United States). In other embodiments, one or more soil characteristics, such as soil texture, soil water holding capacity and soil type, can be used to select the predetermined fixed mass M. Publicly available soil surveys such as USDA SSURGO can be used to make the link between geography and expected soil type/bulk density. In other embodiments, proprietary soil databases can be used to make the link between geography and expected soil type/bulk density. In still further embodiments, the link may be made using publicly available and private, proprietary data sources.

In some embodiments, the intact soil core sample is weighed before the soil is transferred to the second container to determine if the intact soil core sample contains sufficient mass to meet the fixed mass M requirement. If the mass of the intact soil core sample is not sufficient, the process described below is followed.

In some embodiments, the first container may have ruler markings designing the depth of the soil sample. Additionally, or alternatively, the second container may have ruler markings designating the depth of the soil transferred into the second container. In this way, the depth of soil required to satisfy fixed mass M can be determined.

In step 20, a portion or subsample of the analysis sample is provided for analysis to determine the soil organic carbon concentration according to known analysis methods. For example, about 0.1-20 grams, about 0.1-10 grams or about 0.2-5 grams of the analysis sample can be analyzed to determine the soil organic carbon (SOC) concentration. The total organic carbon (TOC) to the depth of the soil that corresponds to reaching the fixed mass M can be calculated by multiplying the SOC concentration by the fixed mass M and this value can be used as the estimate of the SOC stock. In some embodiments, the SOC concentration analysis can be conducted at the testing facility. In other embodiments, the SOC concentration analysis can be conducted at another location.

In some embodiments, the subsample does not include coarse fragments. In some embodiments, the coarse fragments are removed from the analysis sample after reaching fixed mass M and before collecting the subsample. In other embodiments, the coarse fragments are not removed from the analysis sample and are removed from the subsample.

The method described above operates when the fixed mass M is achieved at a soil depth between 30 cm and the depth of the soil core sample. Special treatment is required when the predetermined fixed mass M is not obtained between these depths for the intact soil core delivered to the testing facility.

In many situations, the soil core sample will have a mass equal to or greater than the fixed mass M needed for the analysis sample, and thus, the soil core sample meets the mass requirement. In the situation in which the soil core sample does not have sufficient mass to satisfy the mass requirement (i.e., fixed mass M is achieved at a depth greater than that of the core sample), the actual mass of the analysis sample SM is recorded and is used in place of fixed mass M when calculating the SOC stock in step 18.

As previously discussed, the carbon content can be used as part of a carbon sequestration program in which economic incentives may be given based on the carbon sequestered. Some such programs have protocols which require the carbon content be determined with a sample taken over a specified depth. For example, some programs require that the carbon content be determined with a sample taken over a sufficient depth to characterize changes in soil carbon due to agricultural practices. For example, a carbon protocol may require that the carbon content be determined using at least 30 cm of soil depth. The Intergovernmental Panel on Climate Change (IPCC) states that 30 cm is the minimum depth of sampling required for reliable carbon sequestration. For example, certain agricultural practice changes may cause the carbon to be redistributed along the vertical profile in the soil and not sampling deep enough could make a soil carbon vertical profile redistribution look like a soil carbon change. The situation may arise in which the predetermined fixed mass M for the analysis sample is reached with less depth than is sufficient to characterize changes in soil carbon due to agricultural practices (i.e., less than 30 cm of soil from the soil core sample.) The depth sufficient to characterize changes in soil carbon due to agricultural practices can be referred to as depth DS. In the situation in which the predetermined fixed mass M is reached with less than depth DS, depth DS of soil can be poured from the soil core sample into the analysis sample and the mass of the soil in the analysis sample is recorded. The organic carbon content is determined according to step 20 based on the higher mass.

Prior methods for measuring soil organic carbon were determined by using a fixed volume sample (i.e., to a fixed soil depth) and were based on the product of two independent measurements: (i) bulk density and (ii) soil organic carbon concentration. The method disclosed herein eliminates the bulk density measurement step, instead the method uses a core sample of a fixed mass and measures the soil organic carbon concentration within it. This process has several advantages. First, in some situations, the method can be more accurate in the face of changes in soil bulk density (i.e., soil compaction and expansion), allowing fewer samples to be used to reach the same statistical uncertainty level (i.e., root mean square of prediction) as the fixed volume sample approach. Second, the removal of the need for a bulk density measurement potentially allows for cost savings as the significant statistical measurement uncertainties in bulk density measurement increase the need for more expensive, larger diameter cores.

In the current method, the soil core sample is taken to a specified depth, which is 45 centimeters (cm) in some embodiments. The soil core sample is sent intact to the testing facility where the predetermined fixed mass M is transferred from the intact soil core sample to the second sample container to form the analysis sample. The soil core sample is not segmented into different samples, but rather is sent as a single sample. Further, the method described herein allows the layers of the soil core sample to be retained from the sample site (i.e., agricultural field) to the testing location. Thus, increasing the accuracy of the calculated SOC stock and reducing the cost.

In some embodiments of the current invention, a single core sample is used for all analyses. Certain other prior methods, by comparison, required multiple samples. The reduction from six or seven samples to one sample results in significant cost savings. The reduction in the number of samples in the current process is at least on part because the soil bulk density is not determined. Additionally, the current process results in a reduction of the number of samples because of the increased accuracy of the process, allowing the same statistical uncertainty to be reached with fewer samples.

In some embodiments, the soil is dried, and the fixed mass M is transferred to a second container for the analysis sample. Additional steps, such as removal of coarse fragments from the original sample, are not required. This results in a method with fewer steps to competition and lower cost.

It is to be understood that the above description is intended to be illustrative, and not restrictive. Many other embodiments will be apparent to those of skill in the art upon reading and understanding the above description. It is contemplated that features described in association with one embodiment are optionally employed in addition or as an alternative to features described in or associated with another embodiment. The scope of the invention should, therefore, be determined with reference to the appended claims, along with the full scope of equivalent to which such claims are entitled.

What is claimed is:

1. A method for calculating total organic carbon (TOC) for a sample location, the method comprising:
obtaining an intact core soil sample from a sample location, the core soil sample having a depth and mass;
transferring a predetermined fixed mass M from the intact core soil sample into an analysis sample container to obtain an analysis sample; and
providing a subsample of the analysis sample for soil organic carbon (SOC) concentration testing.

2. The method of claim 1 wherein the predetermined mass is based on a soil characterization of the sample location.

3. The method of claim 2 wherein the soil characterization is soil bulk density, and wherein the predetermined fixed mass M from a sample location having a high soil bulk density is PM1 and the predetermined fixed mass M from a sample location having a low soil bulk density is PM2, where PM1 and PM2 are different.

4. The method of claim 2 wherein the soil characterization is geographic location, and wherein the predetermined fixed mass M from a first geographical location is PM1 and the predetermined fixed mass M from a second geographical location is PM2, where PM1 and PM2 are different.

5. The method of claim 4 wherein a soil database is used to convert the geographic location to a soil bulk density category.

6. The method of claim 1 wherein the depth of the core soil sample is from about 35 centimeters (cm) to about 45 cm.

7. The method of claim 1 wherein the depth of the core soil sample is based on a soil characteristic of the sample location.

8. The method of claim 7 wherein the soil characteristic is soil bulk density.

9. The method of claim 1 wherein the depth of the core soil sample is based on the geographic location of the sample location.

10. The method of claim 1 wherein if the mass of intact core soil sample is less than the predetermined fixed mass M, the predetermined fixed mass M is the mass of the intact core sample.

11. The method of claim 1, wherein if the predetermined fixed mass M is obtained with less than a depth DS, the depth DS from the intact core soil sample is transferred to the analysis sample container with sample mass SM, where DS is the soil depth sufficient to characterize changes in soil carbon due to agriculture practices,

12. The method of claim 11 wherein DS is 30 cm.

13. The method of claim 1 and further including the step of calculating the TOC by multiplying predetermined fixed mass M by a SOC concentration from the SOC concentration testing.
